(19) United States
(12) Patent Application Publication
(10) Pub. No.: US 2013/0066427 A1
(43) Pub. Date: Mar. 14, 2013

(54) Devices and Methods for Tissue Engineering

(71) Applicant: James Jenq Liu, Mason, OH (US)

(72) Inventor: James Jenq Liu, Mason, OH (US)

(73) Assignee: BIO2 TECHNOLOGIES, INC., Woburn, MA (US)

(21) Appl. No.: 13/598,721

(22) Filed: Aug. 30, 2012

Related U.S. Application Data
(60) Provisional application No. 61/532,416, filed on Sep. 8, 2011.

(51) Int. Cl. A61F 2/28; A61F 2/42; B29C 35/02; A61F 2/44

(52) U.S. Cl. 623/17.11; 623/23.61; 623/18.11; 264/42

(57) ABSTRACT

A silicon nitride porous tissue engineering scaffold is fabricated from a silicon-based fiber that is converted to silicon nitride through a reaction at elevated temperatures in a nitrogen environment. Porosity in the form of interconnected pore space is provided by the pore space between the fiber material in a porous matrix. The silicon nitride porous tissue engineering scaffold can be formed from raw materials that are a precursor to silicon nitride. The silicon nitride porous tissue engineering scaffold supports tissue in-growth to provide osteoconductivity as a biocompatible tissue scaffold used as an implantable medical device for the repair of damaged and/or diseased bone tissue.

### CROSS-REFERENCE TO RELATED APPLICATIONS

This application claims priority to U.S. Provision Application Ser. No. 61/532,416 filed Sep. 8, 2011 entitled “Devices and Methods for Tissue Engineering” the content of which is herein incorporated by reference.

### FIELD OF THE INVENTION

The present invention relates generally to the field of porous medical implants. More specifically, the invention relates to a porous fibrous implant having a silicon nitride composition.

### BACKGROUND OF THE INVENTION

Prosthetic devices are often required for repairing defects in bone tissue in surgical and orthopedic procedures. Prostheses are increasingly required for the replacement or repair of diseased or deteriorated bone tissue in an aging population and to enhance the body's own mechanism to produce rapid healing of musculoskeletal injuries resulting from severe trauma or degenerative disease.

Autografting and allografting procedures have been developed for the repair of bone defects. In autografting procedures, bone grafts are harvested from a donor site in the patient, for example from the iliac crest, to implant at the repair site, in order to promote regeneration of bone tissue. However, autografting procedures are particularly invasive, causing risk of infection and unnecessary pain and discomfort at the harvest site. In allografting procedures, bone grafts are used from a donor of the same species but the use of these materials can raise the risk of infection, disease transmission, and immune reactions, as well as religious objections. Accordingly, synthetic materials and methods for implanting synthetic materials have been sought as an alternative to autografting and allografting.

Synthetic prosthetic devices for the repair of defects in bone tissue have been developed in an attempt to provide a material with the mechanical properties of natural bone materials, while promoting bone tissue growth to provide a durable and permanent repair. Knowledge of the structure and bio-mechanical properties of bone, and an understanding of the bone healing process provides guidance on desired properties and characteristics of an ideal synthetic prosthetic device for bone repair. These characteristics include, but are not limited to: biocompatibility so that the device incorporates in the body without harmful side effects; osteostimulation and/or osteoconductivity to promote bone tissue in-growth into the device as the wound heals; and load bearing or weight sharing to support the repair site yet exercise the tissue as the wound heals to promote a durable repair.

Materials developed to date have been successful in attaining at least some of the desired characteristics, but nearly all materials compromise at least some aspect of the bio-mechanical requirements of an ideal hard tissue scaffold.

### BRIEF SUMMARY OF THE INVENTION

The present invention meets the objectives of an effective synthetic bone prosthetic for the repair of bone defects by providing a biocompatible porous structure having a silicon nitride composition. The present invention provides a method of fabricating a porous synthetic bone prosthesis from a mixture of fiber having a silicon-based composition with a bonding agent, pore former, binder, and a liquid. The mixture can be formed into a shaped object and dried. Volatile constituents of the mixture are removed, namely, the pore former, or portions thereof, and the binder, or portions thereof. The shaped object, consisting essentially of overlapping and intertangled fibers with a bonding agent distributed therethrough, is heated in a nitrogen environment to form a silicon nitride composition from the silicon-based fiber and the nitrogen environment having a porosity sufficient to support tissue ingrowth when implanted in living tissue.

In an aspect of the invention the silicon based fiber is a silica fiber. Furthermore, the step of heating the shaped object in a nitrogen environment can be adapted to perform a carbothermal reduction reaction formation of silicon nitride when a carbon constituent is included in the mixture. In an aspect of the invention the bonding agent is silicon nitride powder. In another aspect of the invention the bonding agent is yttrium oxide.

The method of the present invention generally involved a reaction-formation of a silicon nitride composition from silica fibers in a carbothermal reduction reaction using carbon or graphite particles as a pore former. In this method the silica fibers, positioned in an overlapping an intertangled relationship as determined by mixing the fibers with the pore former, are maintained in the same relative position and form upon completion of the reaction-formation of the silicon nitride composition.

These and other features of the present invention will become apparent from a reading of the following descriptions and may be realized by means of the instrumentalities and combination particularly pointed out in the appended claims.

### BRIEF DESCRIPTION OF THE SEVERAL VIEWS OF THE DRAWINGS

The foregoing and other objects, features, and advantages of the invention will be apparent from the following detailed description of the several embodiments of the invention as illustrated in the accompanying drawings in which like reference characters refer to the same parts throughout the different views. The drawings are not necessarily to scale, with emphasis instead being placed upon illustrating the principles of the invention.

FIG. 1 is a scanning electron micrograph at approximately 100× magnification of a representation of an embodiment of a tissue scaffold according to the present invention.

FIG. 2 is a flowchart of an embodiment of a method of the present invention for forming the tissue scaffold of FIG. 1.

FIG. 3 is a flowchart of an embodiment of a curing step according to the method of FIG. 2.

FIG. 4 is a schematic representation of an object fabricated according to a method of the present invention.

FIG. 5 is a schematic representation of the object of FIG. 4 upon completion of a volatile component removal step of the method of the present invention.

FIG. 6 is a schematic representation of the object of FIG. 5 upon completion of a reaction formation step of the method of the present invention.

FIG. 7 is a side elevation view of a tissue scaffold according to the present invention manufactured into a spinal implant.

FIG. 8 is a side perspective view of a spine having the spinal implant of FIG. 7 implanted into the intervertebral space.

FIG. 9 is a schematic drawing showing an isometric view of a tissue scaffold according to the present invention manufactured into an osteotomy wedge.

FIG. 10 is a schematic drawing showing an exploded view of the osteotomy wedge of FIG. 9 operable to be inserted into an osteotomy opening in a bone.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

The present invention provides a synthetic prosthetic tissue scaffold for the repair of tissue defects. In an embodiment, the synthetic prosthetic tissue scaffold is biocompatible once implanted in living tissue. In an embodiment, the synthetic prosthetic tissue scaffold is osteostimulative once implanted in living tissue. In an embodiment, the synthetic prosthetic tissue scaffold is load bearing once implanted in living tissue.

Various types of synthetic materials have been developed or tissue engineering applications in an attempt to provide a synthetic prosthetic device that mimics the properties of natural bone tissue that promotes healing and repair of tissue. Metallic and bio-persistent structures have been developed to provide high strength in a porous structure that can promote the growth of new tissue. These materials are most commonly used in material compositions of medical-grade titanium, tantalum, and stainless steel.

Medical implants of metallic materials exhibit poor radiolucency characteristics that can interfere with post-operative monitoring of the implant. The poor radiolucency of metallic implants presents difficulties in the evaluation of the bone ingrowth due to the radio-shadow produced by the metallic material. In a load-bearing implant the effects of stress shielding are particularly concerning to a clinician that can lead to graft necrosis, poor fusion and instability. Radiographic analysis of the implant is essential to evaluate and diagnose pain and discomfort and to assess the need for revision.

Silicon nitride is a biocompatible ceramic material that is highly chemically and dimensionally stable that has received regulatory clearance for use as an orthopedic implant. An implant of a silicon nitride composition will exhibit radiolucency to permit post-operative monitoring and evaluation. Mechanical properties of implants fabricated using known methods of production using silicon nitride include high strength, high fracture toughness, and high durability with low wear when disposed on an articulating surface. Porosity for osteostimulation and/or bone ingrowth can be provided by surface treatments or coatings on the surface of silicon nitride devices to facilitate ingrowth and adherence of bone tissue.

In an embodiment of the present invention, a porous silicon nitride synthetic prosthetic device is fabricated with an interconnected network of pores that exhibits the superior mechanical characteristics of a silicon nitride material at high porosity (e.g., greater than 40% bulk porosity).

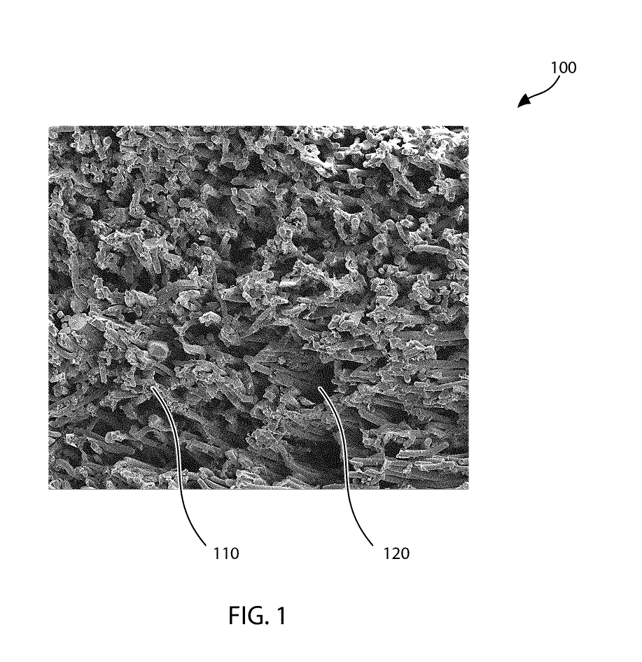

Referring to FIG. 1, a tissue scaffold 100 having a silicon nitride composition according to the present invention is shown. The tissue scaffold 100 is a rigid three-dimensional matrix 110 forming a structure that mimics bone structure in strength and pore morphology. As used herein the term “rigid” means the structure does not significantly yield upon the application of stress until it fracture in the same way that natural bone would be considered to be a rigid structure. The tissue scaffold 100 is a porous material having a network of pores 120 that provide osteoconductivity when implanted in living tissue. As used herein the term osteoconductive means that the material can facilitate the in-growth of bone tissue Cancellous bone of a typical human has a compressive crush strength raging between about 0.1 to about 0.5 GPa. As will be shown herein below, the tissue scaffold 100 of the present invention can provide a porous osteostimulative structure in a silicon nitride composition with porosity greater than about 40% and compressive crush strength greater than 4 MPa, up to, and exceeding 22 MPa.

In an embodiment, the three dimensional matrix 110 is formed from fibers that are bonded and fused into a rigid structure, with a predominate composition of silicon nitride. The use of fibers as a raw material for creating the three dimensional matrix 110 provides a distinct advantage over the use of compacted silicon nitride powders as known in the art. In an embodiment, the fiber-based raw material provides a structure that has more strength at a given porosity than the powder-based materials.

The tissue scaffold 100 of the present invention provides desired mechanical and chemical characteristics combined with pore morphology to promote osteoconductivity. The network of pores 120 of the tissue scaffold 100 is the natural interconnected porosity resulting from the space between intertangled, nonwoven fiber material in a structure that mimics the structure of natural bone. Furthermore, using methods described herein, the pore size can be controlled and optimized to enhance the flow of blood and body fluid within the tissue scaffold 100 and regenerated bone. For example, pore size and pore size distribution can be controlled through the selection of pore formers and organic binders that are volatilized during the formation of the tissue scaffold 100. Pore size and pore size distribution can be determined by the particle size and particle size distribution of the pore former including a single mode of pore sized, a bimodal pore size distribution, and/or a multi-modal pore size distribution. The porosity of the tissue scaffold 100 can be in the range of about 40% to about 85%. In an embodiment, a range of pore size of approximately 200 to 600 μm has been shown to promote the process of osteoinduction of the regenerating tissue once implanted in living tissue while exhibiting load bearing strength.

The tissue scaffold 100 according to the present invention is fabricated using fibers as a raw material that are used to create a silicon nitride composition. The fibers can be composed of silica or a silica-based material that is a precursor silicon nitride. The term “fiber” as used herein is meant to describe a filament or elongated member in a continuous or discontinuous form having an aspect ratio greater then one and formed from a fiber-forming process such as drawn, spun, blown, or other similar process typically used in the formation of fibrous material or high aspect-ratio materials.

Silicon nitride is a ceramic composition that is a chemical compound of silicon and nitrogen (Si₃N₄). The material is thermally and chemically stable in vivo with high strength and a moderately high elastic modulus. Silicon nitride is formed by a reaction between silicon and nitrogen at elevated temperatures (e.g., approximately 1400° C.). The composition can also be formed by carbothermal reduction of silica in a nitrogen atmosphere at temperatures between 1400-1700° C.: 3SiO₂+6C+2N₂→3Si₃N₄+6CO₂.

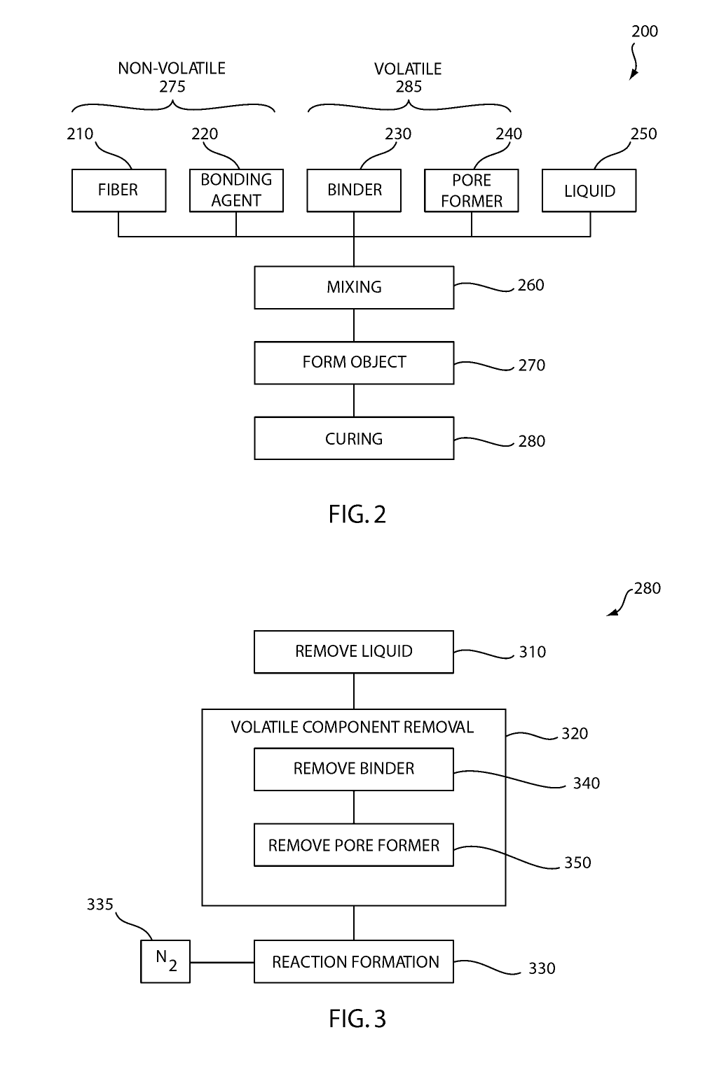

Referring to FIG. 2, an embodiment of a method 200 of forming the tissue scaffold 100 is shown. The method 200 provides for the fabrication of a silicon nitride tissue scaffold using silicon-based fiber materials. Generally, silicon-based fiber is mixed with a bonding agent 200, a binder 230, and a liquid 250 to form a plastically moldable material that is then cured to form the silicon nitride tissue scaffold 100. The curing step 280 selectively removes the volatile elements of the mixture, leaving the pore space 120 open an interconnected and effectively fuses and bonds the fibers 210 into the rigid three-dimensional matrix 110 in a silicon nitride composition.

Bulk fibers 210 can be provided in bulk form or as chopped fibers in a silicon-based or silica-based composition. The fiber 210 can be a silica fiber. The diameter of the fiber 210 can range from about 1 μm to about 200 μm and typically between about 5 to about 100 μm. Fibers 210 of this type can be produced with relatively narrow and controlled distribution of fiber diameters or depending upon the method used to fabricate the fiber, a relatively broad distribution of fiber diameters can be produced. Bulk fibers 210 of a given diameter may be used, or a mixture of fibers having a range of fiber diameters can be used. The diameter of the fibers 210 will influence the resulting pore size, pore size distribution, strength, and elastic modulus of the porous structure, as well as the size and thickness of the three-dimensional matrix 110, which will influence the osteoconductivity of the scaffold 100 when implanted in living tissue and the resulting strength characteristics, including compressive strength and elastic modulus.

The fibers 210 used according to the present invention as herein described are typically continuous and/or chopped silica glass fiber. Silica-based glass in various compositions can be readily drawn into continuous or discontinuous fiber. Examples of fiber 210 that can be used according to the present invention include silica glass or quartz glass fiber. Silica-based compositions with various amounts of alumina, calcium, magnesium and other oxides that form silica-based glass materials. In an embodiment of the invention the fiber 210 is pure silica fiber.

The binder 230 and the liquid 250, when mixed with the fiber 210, create a plastically formable batch mixture that enables the fibers 210 to be evenly distributed throughout the batch, while providing green strength to permit the batch material to be formed into the desired shape in the subsequent forming step 270. An organic binder material can be used as the binder 230, such as methylcellulose, hydroxypropyl methylcellulose (HPMC), ethylcellulose and combinations thereof. The binder 230 can include materials such as polyethylene, polypropylene, polybutene, polystyrene, polyvinyl acetate, polyester, isotactic polypropylene, atactic polypropylene, polysulphone, polyacetal polymers, polymethyl methacrylate, fumaron-indane copolymer, ethylene vinyl acetate copolymer, styrene-butadiene copolymer, acryl rubber, polyvinyl butyral, inomer resin, epoxy resin, nylon, phenol formaldehyde, phenol furfural, paraffin wax, wax emulsions, microcrystalline wax, celluloses, dextrines, chlorinated hydrocarbons, refined alginates, starches, gelatins, lignins, rubbers, acrylics, bitumens, casein, gums, albumins, proteins, glycols, hydroxyethyl cellulose, sodium carboxymethyl cellulose, polyvinyl alcohol, polyvinyl pyrrolidone, polyethylene oxide, polyacrylamides, polyethyterimine, agar, agarose, molasses, dextrines, starch, lignosulfonates, lignin liquor, sodium alginate, gum arabic, xanthan gum, gum tragacanth, gum karaya, locust bean gum, irish moss, scleroglucan, acrylics, and cationic galactomanan, or combinations thereof. Although several binders 230 are listed above, it will be appreciated that other binders may be used. The binder 230 provides the desired rheology and cohesive strength of the plastic batch material in order to form a desired object and maintaining the relative position of the fibers 210 in the mixture while the object is formed, while remaining inert with respect to the silicon nitride precursor materials. The physical properties of the binder 230 will influence the pore size and pore size distribution of the pore space 120 of the scaffold 100. Preferably, the binder 230 is capable of thermal disintegration, or selective dissolution, without impacting the chemical composition of the silicon nitride precursor components, including the fiber 210.

The fluid 250 is added as needed to attain a desired rheology in the plastic batch material suitable for forming the plastic batch material into the desired object in the subsequent forming step 270. Water is typically used, though solvents of various types can be utilized. Rheological measurements can be made during the mixing step 260 to evaluate the plasticity and cohesive strength of the mixture prior to the forming step 270.

Pore formers 240 can be included in the mixture to enhance the pore space 120 of the tissue scaffold 100. Pore formers are non-reactive materials that occupy volume in the plastic batch material during the mixing step 260 and the forming step 270. When used, the particle size and size distribution of the pore former 240 will influence the resulting pore size and pore size distribution of the pore space 120 of the scaffold 100. Particle sizes can typically range between about 25 μm or less to about 450 μm or more, or alternatively, the particle size for the pore former can be a function of the fibers 210 diameter ranging from about 0.1 to about 100 times the diameter of the fibers 210. The pore former 240 must be readily removable during the curing step 280 without significantly disrupting the relative position of the surrounding fibers 210. In an embodiment of the invention, the pore former 240 can be removed via pyrolysis or thermal degradation, or volatilization at elevated temperatures during the curing step 280. For example, microwax emulsions, phenolic resin particles, flour, starch, or carbon particles can be included in the mixture as the pore former 240. Other pore formers 240 can include carbon black, activated carbon, graphite flakes, synthetic graphite, wood flour, modified starch, celluloses, coconut shell husks, latex spheres, bird seeds, saw dust, pyrolyzable polymers, poly (alkyl methacrylate), polymethyl methacrylate, polyethyl methacrylate, poly n-butyl methacrylate, polyethers, poly tetrahydrofuran, poly (1, 3-dioxolane), poly (alkalene oxides), polyethylene oxide, polypropylene oxide, methacrylate copolymers, polyisobutylene, polytrimethylene carbonate, polyethylene oxalate, polybeta-propiolactone, polydelta-valerolactone, polyethylene carbonate, polypropylene carbonate, vinyl toluene/alpha-methylstyrene copolymer, styrene/alpha-methyl styrene copolymers, and olefin-sulfur dioxide copolymers. Pore formers 240 may be generally defined as organic or inorganic, with the organic typically burning off at a lower temperature than the inorganic. Although several pore formers 240 are listed above, it will be appreciated that other pore formers 240 may be used. Pore formers 240 can be, though need not be, fully biocompatible since they are removed from the scaffold 100 during processing.

Additional materials in powder or particle-based form can be provided as a bonding agent 220 to combine with the fiber 210 to form the silicon nitride composition of the three-dimensional matrix 110 and to promote the strength and performance of the resulting tissue scaffold 100. The bonding agent 220 can include powder-based silicon nitride material, or it can contain powder-based or colloidal silica as a precursor to the silicon nitride composition. The bonding agent 220 can include sintering aids that promote localized liquid-phase reaction during the formation of silicon nitride or materials that can enhance the efficiency of the carbothermal reduction reaction of silica to silicon nitride. For example, the bonding agent 220 can include small quantities of yttrium oxide (Y₂O₃) due to its affinity for oxygen at elevated temperatures. In an embodiment of the invention the bonding agent 220 can be coated on the fibers 210 as a sizing or coating. In this embodiment, additional precursors to the silicon nitride composition are added to the fiber, for example, as a sizing or coating. In an alternate embodiment, the bonding agent 220 is a sizing or coating that is added to the fiber during or prior to the mixing step 260. The bonding agent 220 can be solids dissolved in a solvent or liquid that are deposited on the fiber and/or other bonding agent 220 precursors when the liquid or solvent is removed. As will be explained in further detail below, the bonding agent 220 based additives enhance the bonding strength of the intertangled fibers 210 forming the three-dimensional matrix 110 through the formation of bonds between adjacent and intersecting fibers 210 when the bonding agent 220 reacts with the fiber 210 to form the desired silicon nitride composition. The relative quantities of the fiber 210 and the bonding agent 220 generally determine the resulting composition of the three-dimensional matrix 110.

The relative quantities of the respective materials, including the bulk fiber 210, the binder 230, and the liquid 250 depend on the overall porosity desired in the tissue scaffold 100. For example, to provide a scaffold 100 having approximately 60% porosity, the nonvolatile components 275, such as the fiber 210, would amount to approximately 40% of the mixture by volume. The relative quantity of volatile components 285, such as the binder 230 and the liquid 250 would amount to approximately 60% of the mixture by volume, with the relative quantity of binder to liquid determined by the desired rheology of the mixture. Furthermore, to produce a scaffold 100 having porosity enhance by the pore former 240, the amount of the volatile components 285 is adjusted to include the volatile pore former 240. Similarly, to produce a scaffold 100 having strength enhanced by the bonding agent 220, the amount of the nonvolatile components 275 would be adjusted to include the nonvolatile bonding agent 220. It can be appreciated that the relative quantities of the nonvolatile components 275 and volatile components 285 and the resulting porosity of the scaffold 100 will vary as the material density may vary due to the reaction of the components during the curing step 280. Specific examples are provided herein below.

In the mixing step 260, the fiber 210, the binder 230, the liquid 250, the pore former 240 and/or the bonding agent 220, if included, are mixed into a homogeneous mass of a plastically deformable and uniform mixture. The mixing step 260 may include dry mixing, wet mixing, shear mixing, and kneading, which can be necessary to evenly distribute the material into a homogeneous mass while imparting the requisite shear forces to break up and distribute or de-agglomerate the fibers 210 with the non-fiber materials. The amount of mixing, shearing, and kneading, and duration of such mixing processes depends on the selection of fibers 210 and non-fiber materials, along with the selection of the type of mixing equipment used during the mixing step 260, in order to obtain a uniform and consistent distribution of the materials within the mixture, with the desired rheological properties for forming the object in the subsequent forming step 270. Mixing can be performed using industrial mixing equipment, such as batch mixers, shear mixers, and/or kneaders. The kneading element of the mixing step 260 distributes the fiber 210 with the bonding agent 220 and the binder 230 to provide a formable batch of a homogeneous mass with the fiber being arranged in an overlapping and intertangled relationship with the remaining fiber in the batch.

The forming step 270 forms the mixture from the mixing step 260 into the object that will become the tissue scaffold 100. The forming step 270 can include extrusion, rolling, pressure casting, or shaping into nearly any desired form in order to provide a roughly shaped object that can be cured in the curing step 280 to provide the scaffold 100. It can be appreciated that the final dimensions of the scaffold 100 may be different than the formed object at the forming step 270, due to expected shrinkage of the object during the curing step 280, and further machining and final shaping may be necessary to meet specified dimensional requirements. In an exemplary embodiment to provide samples for mechanical and in vitro and in vivo testing, the forming step 270 extrudes the mixture into a cylindrical rod using a piston extruder forcing the mixture through a round die.

The object is then cured into the tissue scaffold 100 in the curing step 280, as further described in reference to FIG. 3. In the embodiment shown in FIG. 3, the curing step 280 can be performed as the sequence of three phases: a drying step 310; a volatile component removal step 320; and a reaction formation step 330. In the first phase, drying 310, the formed object is dried by removing the liquid using slightly elevated temperature heat with or without forced convection to gradually remove the liquid. Various methods of heating the object can be used, including, but not limited to, heated air convection heating, vacuum freeze drying, solvent extraction, microwave or electromagnetic/radio frequency (RF) drying methods. The liquid within the formed object is preferably not removed too rapidly to avoid drying cracks due to shrinkage. Typically, for aqueous based systems, the formed object can be dried when exposed to temperatures between about 90° C. and about 150° C. for a period of about one hour, though the actual drying time may vary due to the size and shape of the object, with larger, more massive objects taking longer to dry. In the case of microwave or RF energy drying, the liquid itself, and/or other components of the object, adsorb the radiated energy to more evenly generate heat throughout the material. During the drying step 310, depending on the selection of materials used as the volatile components, the binder 230 can congeal or gel to provide greater green strength to provide rigidity and strength in the object for subsequent handling.

Once the object is dried, or substantially free of the liquid component 250 by the drying step 310, the next phase of the curing step 280 proceeds to the volatile component removal step 320. This phase removes the volatile components (e.g., the binder 230 and the pore former 240) from the object leaving only the non-volatile components that form the three-dimensional matrix 110 of the tissue scaffold 100. The volatile components can be removed, for example, through pyrolysis or by thermal degradation, or solvent extraction. The volatile component removal step 320 can be further parsed into a sequence of component removal steps, such as a binder burnout step 340 followed by a pore former removal step 350, when the volatile components 285 are selected such that the volatile component removal step 320 can sequentially remove the components. For example, HPMC used as a binder 230 will thermally decompose at approximately 300° C. A graphite pore former 220 will oxidize into carbon dioxide when heated to approximately 600° C. in the presence of oxygen. Similarly, flour or starch, when used as a pore former 220, will thermally decompose at temperatures between about 300° C. and about 600° C. Accordingly, the formed object composed of a binder 230 of HPMC and a pore former 220 of graphite particles, can be processed in the volatile component removal step 320 by subjecting the object to a two-step firing schedule to remove the binder 230 and then the pore former 220. In this example, the binder burnout step 340 can be performed at a temperature of at least about 300° C. but less than about 600° C. for a period of time. The pore former removal step 350 can then be performed by increasing the temperature to at least about 600° C. with the inclusion of oxygen into the heating chamber. This thermally-sequenced volatile component removal step 320 provides for a controlled removal of the volatile components 285 while maintaining the relative position of the non-volatile components 275 in the formed object.

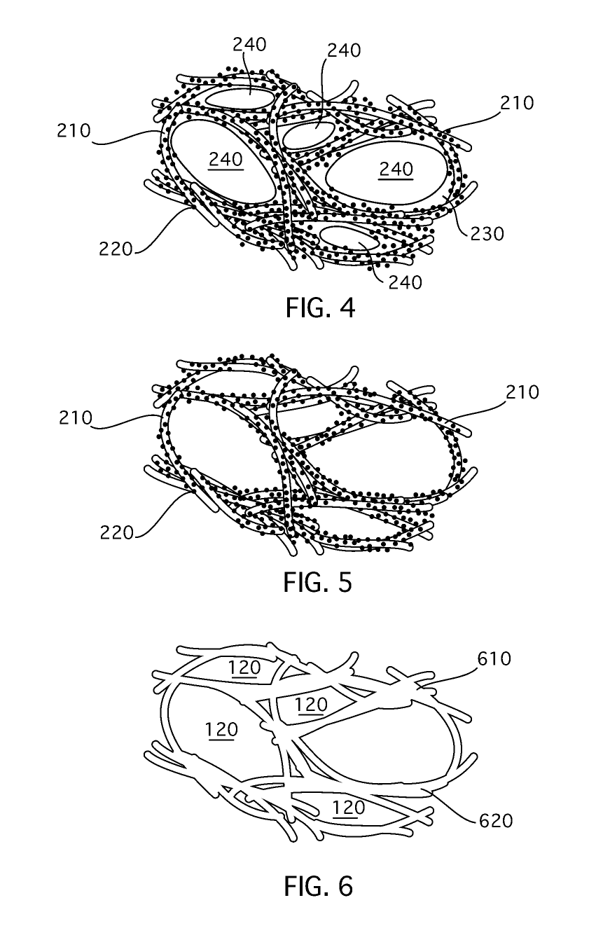

FIG. 4 depicts a schematic representation of the various components of the formed object prior to the volatile component removal step 320. The fibers 210 are intertangled within a mixture of the bonding agent 220, binder 230 and the pore former 240. FIG. 5 depicts a schematic representation of the formed object upon completion of the volatile component removal step 320. The fibers 210 and bonding agent 220 maintain their relative position as determined from the mixture of the fibers 210 with the volatile components 285 as the volatile components 285 are removed. Upon completion of the removal of the volatile components 285, the mechanical strength of the object may be somewhat fragile, and handling of the object in this state should be performed with care. In an embodiment, each phase of the curing step 280 is performed in the same oven or kiln. In an embodiment, a handling tray is provided upon which the object can be processed to minimize handling damage.

FIG. 6 depicts a schematic representation of the formed object upon completion of the last step of the curing step 280, reaction formation 330. Pore space 120 is created between the bonded and intertangled fibers where the binder 230 and the pore former 240 were removed, and the fibers 210 and bonding agent 220 are fused and bonded into the three dimensional matrix 110. The characteristics of the volatile components 285, including the size of the pore former 240 and/or the distribution of particle sizes of the pore former 240 and/or the relative quantity of the binder 230, together cooperate to predetermine the resulting pore size, pore size distribution, and pore interconnectivity of the resulting tissue scaffold 100. The bonding agent 220 and the bonds that form at overlapping nodes 610 and adjacent nodes 620 of the three dimensional matrix 110 provide for structural integrity of the resulting three-dimensional matrix 110 having a bioactive composition.

Referring back to FIG. 3, the reaction formation step 330 reacts the nonvolatile components 275, including the bulk fiber 210, into the rigid three-dimensional matrix 110 having a silicon nitride composition as the tissue scaffold 100 while maintaining the pore space 120 created by the removal of the volatile components 275 and maintaining the relative positioning of the fiber 210. The reaction formation step 330 introduces nitrogen 335 in a chamber and heats the non-volatile components 275 to a temperature upon which the bulk fibers 210 having a silicon-based composition react with the nitrogen environment 335 to form silicon nitride and bond to adjacent and overlapping fibers 210, and for a duration sufficient for the reaction to occur and to form the bonds without melting the fibers 210 or otherwise destroying or diminishing the relative positioning of the non-volatile components 275. The reaction and bond formation temperature and duration depends on the chemical composition and relative size of the fiber 210.

In an embodiment of the invention the fiber 210 comprises silica and the reaction formation step 330 is a carbothermal reduction of the silica fiber in the nitrogen environment to form silicon nitride. In this embodiment carbon or graphite particles are included as a bonding agent, though volatile and consumed during the reaction formation step 330. In this embodiment the pore former selection and subsequent removal at step 350 must selectively retain the carbon or graphite material. In an alternate embodiment of the invention the fiber 210 comprises silica and the reaction formation step 330 is a carbothermal reduction of the silica fiber and carbon or graphite particles are provided as a pore former 240. In this alternate embodiment the pore former removal step 350 occurs substantially at the same time as the reaction formation step 330 wherein substantially all the pore former 240 is consumed during the reaction formation step 330.

### EXAMPLES

The following examples are provided to further illustrate and to facilitate the understanding of the disclosure. These specific examples are intended to be illustrative of the disclosure and are not intended to be limiting in any way.

In a first exemplary embodiment a scaffold is formed from silica fiber by mixing 12.86 grams silica fiber having an average diameter of approximately 30 μm chopped into lengths of approximately 1 to 3 mm, in bulk form with 1 gram of silicon nitride powder as the bonding agent, the fiber and bonding agent comprising the non-volatile components, with 7 grams carbon black powder as the pore former and 5 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,600° C. for 8 hours to convert the silica fiber to silicon nitride.

In a second exemplary embodiment a scaffold is formed from silica fiber by mixing 6.43 grams silica fiber having an average diameter of approximately 30 μm chopped into lengths of approximately 1 to 3 mm, in bulk form with 2 grams of silicon nitride powder and 1 gram yttrium oxide as the bonding agent, the fiber and bonding agent comprising the non-volatile components, with 4 grams carbon black powder as the pore former and 2 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,600° C. for 8 hours to convert the silica fiber to silicon nitride.

In a third exemplary embodiment a scaffold is formed from silica fiber by mixing 6.43 grams silica fiber having an average diameter of approximately 30 μm chopped into lengths of approximately 1 to 3 mm, in bulk form with 2 grams of silicon nitride powder and 1 gram yttrium oxide as the bonding agent, the fiber and bonding agent comprising the non-volatile components, with 4 grams carbon black powder and 4 grams graphite particles as the pore former and 2 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,700° C. for 10 hours to convert the silica fiber to silicon nitride.

In a fourth exemplary embodiment a scaffold is formed from silica fiber by mixing 6.43 grams silica fiber having an average diameter of approximately 30 μm chopped into lengths of approximately 1 to 3 mm, in bulk form with 4 grams of silicon nitride powder and 2 grams yttrium oxide as the bonding agent, the fiber and bonding agent comprising the non-volatile components, with 4 grams carbon black powder and 2 grams graphite particles as the pore former and 2 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,700° C. for 10 hours to convert the silica fiber to silicon nitride.

In a fifth exemplary embodiment a scaffold is formed from silica fiber by mixing 6.43 grams silica fiber having an average diameter of approximately 30 um chopped into lengths of approximately 1 to 3 mm, in bulk form with 0.5 grams of silicon nitride powder and 0.5 grams yttrium oxide as the bonding agent, the fiber and bonding agent comprising the non-volatile components, with 2.57 grams carbon black powder as the pore former and 2 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,700° C. for 10 hours to convert the silica fiber to silicon nitride.

In a sixth exemplary embodiment a scaffold is formed from silica fiber by mixing 6.43 grams silica fiber having an average diameter of approximately 30 μm chopped into lengths of approximately 1 to 3 mm, in bulk form with 1 gram of silicon nitride powder and 1 gram yttrium oxide as the bonding agent, the fiber and bonding agent comprising the non-volatile components, with 4 grams carbon black powder as the pore former and 2 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,700° C. for 10 hours to convert the silica fiber to silicon nitride.

In a seventh exemplary embodiment a scaffold is formed from silica fiber by mixing 6.43 grams silica fiber having an average diameter of approximately 30 μm chopped into lengths of approximately 1 to 3 mm, in bulk form with 1 gram of silicon nitride powder and 0.5 grams yttrium oxide as the bonding agent, the fiber and bonding agent comprising the non-volatile components, with 4 grams carbon black powder as the pore former and 2 grams HPMC as the binder and deionized water to create a plastically formable mixture. The mixture was compression-molded into a 14 mm diameter rod and dried in a convection oven. The part was heated in a nitrogen-purged furnace using a heating profile with a hold at approximately 350° C. to remove the binder and 1,700° C. for 10 hours to convert the silica fiber to silicon nitride.

The tissue scaffolds of the present invention can be used in procedures such as an osteotomy (for example in the hip, knee, hand and jaw), a repair of a structural failure of a spine (for example, an intervertebral prosthesis, lamina prosthesis, sacrum prosthesis, vertebral body prosthesis and facet prosthesis), a bone defect filler, fracture revision surgery, tumor resection surgery, hip and knee prostheses, bone augmentation, dental extractions, long bone arthrodesis, ankle and foot arthrodesis, including subtalar implants, and fixation screws pins. The tissue scaffolds of the present invention can be used in the long bones, including, but not limited to, the ribs, the clavicle, the femur, tibia, and fibula of the leg, the humerus, radius, and ulna of the arm, metacarpals and metatarsals of the hands and feet, and the phalanges of the fingers and toes. The tissue scaffolds of the present invention can be used in the short bones, including, but not limited to, the carpals and tarsals, the patella, together with the other sesamoid bones. The tissue scaffolds of the present invention can be used in the other bones, including, but not limited to, the cranium, mandible, sternum, the vertebrae and the sacrum. In an embodiment, the tissue scaffolds of the present invention have high load bearing capabilities compared to conventional devices. In an embodiment, the tissue scaffolds of the present invention require less implanted material compared to conventional devices. Furthermore, the use of the tissue scaffold of the present invention requires less ancillary fixation due to the strength of the material. In this way, the surgical procedures for implanting the device are less invasive, more easily performed, and do not require subsequent surgical procedures to remove instruments and ancillary fixations.

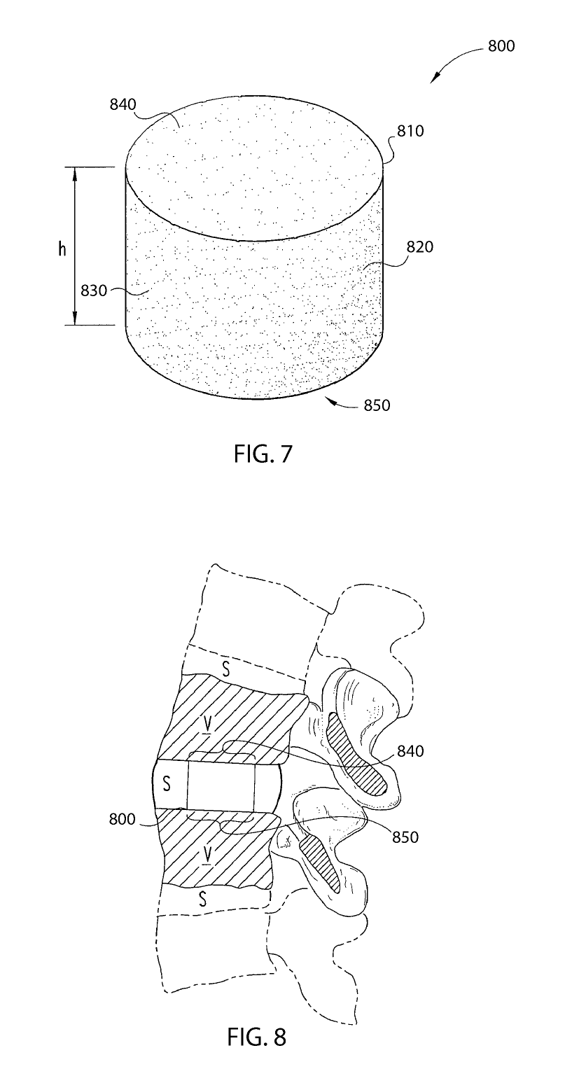

In one specific application, a tissue scaffold of the present invention, fabricated as described above, can be used as a spinal implant 800 as depicted in FIG. 7 and FIG. 8. Referring to FIG. 7 and FIG. 8, the spinal implant 800 includes a body 810 having a wall 820 sized for engagement within a space S between adjacent vertebrae V to maintain the space S. The device 800 is formed from bioactive glass fibers that can be formed into the desired shape using extrusion methods to form a cylindrical shape that can be cut or machined into the desired size. The wall 820 has a height h that corresponds to the height H of the space S. In one embodiment, the height h of the wall 820 is slightly larger than the height H of the intervertebral space S. The wall 820 is adjacent to and between a superior engaging surface 840 and an inferior engaging surface 850 that are configured for engaging the adjacent vertebrae V as shown in FIG. 8.

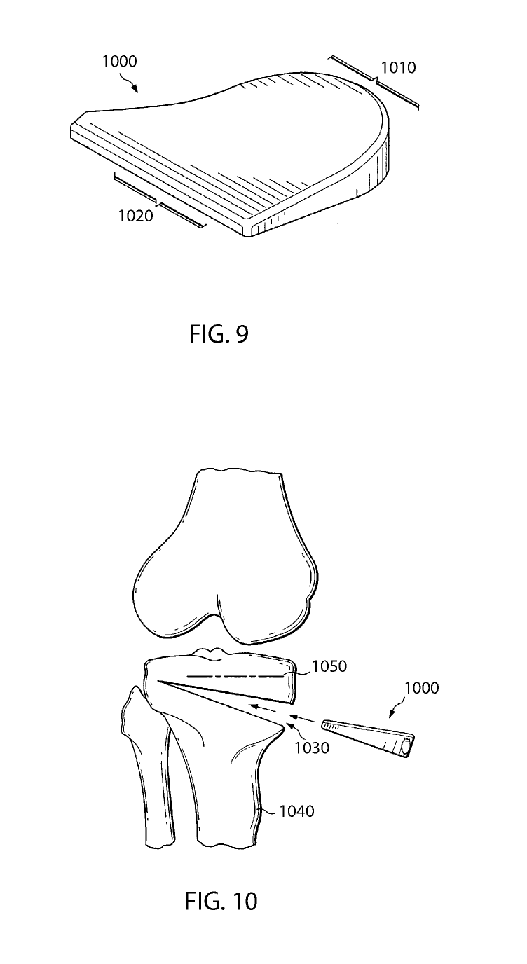

In another specific application, a tissue scaffold of the present invention, fabricated as described above, can be used as an osteotomy wedge implant 1000 as depicted in FIG. 9 and FIG. 10. Referring to FIG. 9 and FIG. 10, the osteotomy implant 1000 may be generally described as a wedge designed to conform to an anatomical cross section of, for example, a tibia, thereby providing mechanical support to a substantial portion of a tibial surface. The osteotomy implant is formed from bioactive glass fibers bonded and fused into a porous material that can be formed from an extruded rectangular block, and cut or machined into the contoured wedge shape in the desired size. The proximal aspect 1010 of the implant 1000 is characterized by a curvilinear contour. The distal aspect 1020 conforms to the shape of a tibial bone in its implanted location. The thickness of the implant 1000 may vary from about five millimeters to about twenty millimeters depending on the patient size and degree of deformity. Degree of angulation between the superior and inferior surfaces of the wedge may also be varied.

FIG. 10 illustrates one method for the use of the osteotomy wedge implant 1000 for realigning an abnormally angulated knee. A transverse incision is made into a medial aspect of a tibia while leaving a lateral portion of the tibia intact and aligning the upper portion 1050 and the lower portion 1040 of the tibia at a predetermined angle to create a space 1030. The substantially wedge-shaped implant 1000 is inserted in the space 1030 to stabilize portions of the tibia as it heals into the desired position with the implant 1000 dissolving into the body as herein described. Fixation pins may be applied as necessary to stabilize the tibia as the bone regenerates and heals the site of the implant.

Generally, the use of a tissue scaffold of the present invention as a bone graft involves surgical procedures that are similar to the use of autograft or allograft bone grafts. The bone graft can often be performed as a single procedure if enough material is used to fill and stabilize the implant site. In an embodiment, fixation pins can be inserted into the surrounding natural bone, and/or into and through the tissue scaffold. The tissue scaffold is inserted into the site and fixed into position. The area is then closed up and after a certain healing and maturing period, the bone will regenerate and become solidly fused.

The use of a tissue scaffold of the present invention as a bone defect filler involves surgical procedures that can be performed as a single procedure, or multiple procedures in stages or phases of repair. In an embodiment, a tissue scaffold of the present invention is placed at the bone defect site, and attached to the bone using fixation pins or screws. Alternatively, the tissue scaffold can be externally secured into place using braces. The area is then closed up and after a certain healing and maturing period, the bone will regenerate to repair the defect.

A method of filling a defect in a bone includes filling a space in the bone with a tissue scaffold in a silicon nitride composition of fibers bonded into a porous matrix, the porous matrix having a pore size distribution to facilitate in-growth of bone tissue; and attaching the tissue scaffold to the bone.

A method of treating an osteotomy includes filling a space in the bone with a tissue scaffold in a silicon nitride composition of fibers bonded into a porous matrix, the porous matrix having a pore size distribution to facilitate in-growth of bone tissue; and attaching the tissue scaffold to the bone.

A method of treating a structural failure of a vertebrae includes filling a space in the bone with a tissue scaffold in a silicon nitride composition of fibers bonded into a porous matrix, the porous matrix having a pore size distribution to facilitate in-growth of bone tissue; and attaching the tissue scaffold to the bone.

In an embodiment, the present invention discloses the use of precursors to form a porous matrix having a silicon nitride composition through a chemical reaction that leads to the transformation of one set of chemical substances (the precursors) to another chemical substance (the silicon nitride composition). The reaction forms at elevated temperatures that is sustained over a period of time.

In an embodiment, the present invention discloses use of fibers bonded into a porous matrix having a silicon nitride composition, the porous matrix having a pore size distribution to facilitate in-growth of bone tissue for the treatment of a bone defect.

In an embodiment, the present invention discloses use of fibers bonded into a porous matrix having a silicon nitride composition, the porous matrix having a pore size distribution to facilitate in-growth of bone tissue for the treatment of an osteotomy.

In an embodiment, the present invention discloses use of fibers bonded into a porous matrix having a silicon nitride composition, the porous matrix having a pore size distribution to facilitate in-growth of bone tissue for the treatment of a structural failure of various parts of a spinal column.

The present invention has been herein described in detail with respect to certain illustrative and specific embodiments thereof, and it should not be considered limited to such, as numerous modifications are possible without departing from the spirit and scope of the appended claims.

What is claimed is:

1. A method of fabricating a porous synthetic bone prosthesis comprising:
mixing a silicon-based fiber with a bonding agent, a pore former, a binder, and a liquid to provide a plastically formable batch, the silicon-based fiber having an intertangled and overlapping relationship;
forming the plastically formable batch into a shaped object;
drying the shaped object by removing substantially all the liquid;
removing the binder and the pore former wherein the intertangled and overlapping relationship is substantially maintained; and
heating the shaped object in a nitrogen environment to react the silicon-based fiber with the nitrogen to form a silicon nitride composition having a porosity to support tissue ingrowth.

2. The method according to claim 1 wherein the silicon-based fiber comprises silica.

3. The method according to claim 2 wherein the mixing step includes carbon and wherein the step of heating the shaped object in a nitrogen environment comprises a carbothermal reduction of the silica using the carbon.

4. The method according to claim 2 wherein the pore former comprises carbon particles wherein the step of heating the shaped object in a nitrogen environment comprises a carbothermal reduction of the silica using the pore former.

5. The method according to claim 1 wherein the bonding agent includes silicon nitride particles.

6. The method according to claim 1 wherein the bonding agent includes yttrium oxide.

7. The method according to claim 1 wherein the bonding agent is in the form of a coating on the silicon-based fiber.

8. The method according to claim 3 wherein the silicon-based fiber is a silica quartz glass.

9. A synthetic bone prosthesis comprising:
intertangled and overlapping fibers bonded into a rigid three-dimensional matrix, the rigid three-dimensional matrix having a silicon nitride composition;
a bulk porosity in the range of about 40% to about 70%; and
a pore size distribution in the rigid three-dimensional matrix with a mode in the range of about 200-600 μm.

10. The synthetic bone prosthesis according to claim 9 wherein the pore size distribution in the rigid three-dimensional matrix has a mode in the range of about 50 μm.

11. The synthetic bone prosthesis according to claim 9 adapted for use as a intervertebral device.

12. The synthetic bone prosthesis according to claim 9 adapted for use as an osteotomy wedge.

13. The synthetic bone prosthesis according to claim 9 adapted for use as a bone graft.

14. The synthetic bone prosthesis according to claim 9 adapted for use as a bone defect filler.

15. The synthetic bone prosthesis according to claim 9 adapted for use as a subtalar implant.
